(19) United States
(12) Patent Application Publication
(10) Pub. No.: US 2026/0334912 A1
(43) Pub. Date: Oct. 29, 2026

(54) APPARATUS AND METHOD FOR CONTROLLING A ROBOTIC FRAMEWORK TO PERFORM IMAGING MODALITIES

(71) Applicant: Anumana, Inc., Cambridge, MA (US)

(72) Inventors: Rakesh Barve, Bengaluru (IN); Abhijith Chunduru, Bengaluru (IN)

(73) Assignee: Anumana, Inc., Cambridge, MA (US)

(21) Appl. No.: 19/188,942

(22) Filed: Apr. 24, 2025

(51) Int. Cl. A61B 8/00; A61B 8/12

(52) U.S. Cl. CPC A61B 8/4218; A61B 8/12; A61B 8/4254; A61B 8/466; A61B 8/54

(57) ABSTRACT

An apparatus and method for controlling a robotic framework to perform imaging modalities. The apparatus includes at least a processor and a memory communicatively connected to the at least a processor. The memory instructs the processor to receive a target region, receive, from a localization system, the at least a position signal of the catheter, generate, using at least a reinforcement-learning model, a movement vector as a function of the at least a position signal, control, using the at least a processor, the robotic framework, as a function of the movement vector, and generate, as a function of the plurality of ultrasonic signals, a three-dimensional (3D) model of the target region.

### FIELD OF THE INVENTION

The present invention generally relates to the field of autonomous robotics. In particular, the present invention is directed to an apparatus and a method for controlling a robotic framework to perform imaging modalities.

### BACKGROUND

In the field of medical imaging, accurately navigating catheters within a patient's body remains a significant challenge, especially when targeting complex anatomical regions for diagnostic or therapeutic purposes. Existing methods often lack real-time adaptability and precision, leading to inefficiencies in imaging workflows and suboptimal outcomes.

### SUMMARY OF THE DISCLOSURE

In an aspect, an apparatus for controlling a robotic framework to perform imaging modalities includes at least a processor and a memory communicatively connected to the at least a processor. The memory contains instructions configuring the processor to receive a target region, receive, from the localization system, the at least a position signal of the catheter, generate, using at least a reinforcement-learning model, a movement vector as a function of the at least a position signal, control, using the at least a processor, the robotic framework, as a function of the movement vector, and generate, as a function of the plurality of ultrasonic signals, a three-dimensional (3D) model of the target region.

In another aspect, a method for controlling a robotic framework to perform imaging modalities includes receiving, using the at least a processor, a target region, receiving, from a localization system, the at least a position signal of the catheter, wherein the localization system is configured to detect at least a position signal as a function of a location of a catheter, generating, using at least a reinforcement-learning model, a movement vector as a function of the at least a position signal, controlling, using the at least a processor, a robotic framework, as a function of the movement vector, wherein the robotic framework is operatively connected to a catheter coupled to at least a transducer, wherein the at least a transducer is configured to receive a plurality of ultrasonic signals, and generating, as a function of the plurality of ultrasonic signals, a three-dimensional (3D) model of the target region.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an apparatus for controlling a robotic framework to perform imaging modalities;

FIG. 2 illustrates a schematic diagram of an exemplary transesophageal echocardiogram;

FIG. 3 is a flow diagram of an exemplary embodiment of an ICE image example generation process;

FIG. 4 is a block diagram of an exemplary machine-learning process;

FIG. 5 is a diagram of an exemplary embodiment of a neural network;

FIG. 6 is a diagram of an exemplary embodiment of a node of a neural network;

FIG. 7 is a block diagram of an exemplary method for controlling a robotic framework to perform imaging modalities; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for controlling a robotic framework to perform imaging modalities. The apparatus includes a robotic framework, wherein the robotic framework operatively connected to a catheter coupled to at least a transducer, wherein the at least a transducer is configured to receive a plurality of ultrasonic signals, at least a localization system configured to detect at least a position signal as a function of a location of the catheter, and at least a computing device comprised of a processor and a memory communicatively connected to the processor. The memory instructs the processor to receive a target region. The processor receives, from the localization system, the at least a position signal of the catheter. The processor generates, using at least a reinforcement-learning model, a movement vector as a function of the at least a position signal. Additionally, the processor control the robotic framework, as a function of the movement vector. The memory then instructs the processor to generate, as a function of the plurality of ultrasonic signals, a three-dimensional (3D) model of the target region.

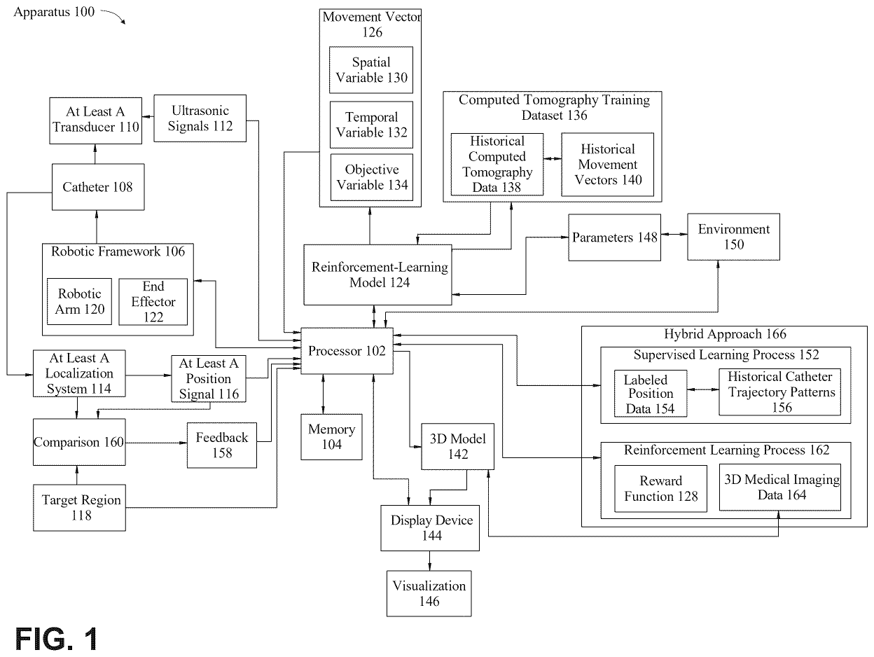

Referring now to FIG. 1, an exemplary embodiment of apparatus 100 for controlling a robotic framework 106 to perform imaging modalities is illustrated. Apparatus 100 may include a processor 102 communicatively connected to a memory 104. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 104 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 102 may access the information from primary memory.

Still referring to FIG. 1, apparatus 100 may include a database. The database may include a remote database. The database may be implemented, without limitation, as a relational database, a key-value retrieval database such as a NOSQL database, or any other format or structure for use as database that a person skilled in the art would recognize as suitable upon review of the entirety of this disclosure. The database may alternatively or additionally be implemented using a distributed data storage protocol and/or data structure, such as a distributed hash table or the like. The database may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, apparatus 100 may include and/or be communicatively connected to a server, such as but not limited to, a remote server, a cloud server, a network server and the like. In one or more embodiments, the computing device may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by the apparatus computing device. In one or more embodiments, computing device may transmit processes to server wherein computing device may conserve power or energy.

Further referring to FIG. 1, apparatus 100 may include any “computing device” as described in this disclosure, including without limitation a microcontroller, microprocessor, digital signal processor (DSP) and/or system on a chip (SoC) as described in this disclosure. Apparatus 100 may include, be included in, and/or communicate with a mobile device such as a mobile telephone or smartphone. Apparatus 100 may include a single computing device operating independently, or may include two or more computing devices operating in concert, in parallel, sequentially or the like; two or more computing devices may be included together in a single computing device or in two or more computing devices. Apparatus 100 may interface or communicate with one or more additional devices as described below in further detail via a network interface device. Network interface device may be utilized for connecting processor 102 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (eg., the Internet, an enterprise network), a local area network (eg., a network associated with an office, a building, a campus or other relatively small geographic space), a telephone network, a data network associated with a telephone/voice provider (eg., a mobile communications provider data and/or voice network), a direct connection between two computing devices, and any combinations thereof. A network may employ a wired and/or a wireless mode of communication. In general, any network topology may be used. Information (eg., data, software etc.) may be communicated to and/or from a computer and/or a computing device. Processor 102 may include but is not limited to, for example, a computing device or cluster of computing devices in a first location and a second computing device or cluster of computing devices in a second location. Apparatus 100 may include one or more computing devices dedicated to data storage, security, distribution of traffic for load balancing, and the like. Apparatus 100 may distribute one or more computing tasks as described below across a plurality of computing devices of computing device, which may operate in parallel, in series, redundantly, or in any other manner used for distribution of tasks or memory between computing devices. Apparatus 100 may be implemented, as a non-limiting example, using a “shared nothing” architecture.

With continued reference to FIG. 1, processor 102 may be designed and/or configured to perform any method, method step, or sequence of method steps in any embodiment described in this disclosure, in any order and with any degree of repetition. For instance, processor 102 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 102 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, the apparatus includes a robotic framework 106, wherein the robotic framework 106 operatively connected to a catheter 108 coupled to at least a transducer 110, wherein the at least a transducer 110 is configured to receive a plurality of ultrasonic signals 112. As used in this disclosure, a “robotic framework” is a system comprising one or more robotic components configured to perform a task. In an embodiment, the robotic framework 106 may be communicatively connected to the at least a processor 102. In an embodiment, the robotic components may include robotic arms, end effectors, manipulators, and the like. In an embodiment, the task may be automated or semi-automated. The robotic framework 106 may include control systems, sensors, actuators, communication interfaces, and the like, to enable precise movement, interaction with the environment 150, and execution of predefined or dynamically generated operations. In a non-limiting example, the robotic framework 106 may include a multi-axis robotic arm 120 equipped with encoders integrated into its joints or actuators. These encoders may provide precise positional or angular feedback to enable accurate movement and control of the arm. For instance, encoders may allow the system to track the orientation and position of the robotic framework 106 in real-time, ensuring precise navigation when performing tasks such as catheter 108 insertion, target region 118 localization, or imaging alignment. This positional data may be used in conjunction with a computing device to adjust the movements of the robotic framework 106 dynamically based on environmental feedback or a predefined trajectory.

With continued reference to FIG. 1, the robotic framework 106 may be configured to perform ultrasonic imaging (echocardiography) of the heart to generate a 3D model 142. The robotic framework 106 can utilize this 3D model 142 at various stages of a medical intervention, including prior to, during, or after surgical procedures. These procedures may include, but are not limited to, ablation, electrophysiology (EP) procedures, left atrial appendage device (e.g., Watchman) placement, valve repair or replacement, and stent placement. As used in this disclosure, “ablation” refers to a medical procedure that removes or destroys abnormal tissue within the body. Without limitation, ablation may be performed using techniques such as radiofrequency energy, cryotherapy, laser energy, and the like. As used in this disclosure, “electrophysiology (EP)” is the study and treatment of the electrical activities of the heart. EP procedures may include diagnostic and therapeutic techniques, such as mapping the heart's electrical pathways and performing ablation to address arrhythmias, ensuring proper heart rhythm. As used in this disclosure, “Watchman placement” is a procedure for implanting a left atrial appendage closure device, known as the Watchman device. As used in this disclosure, “valve repair” is a surgical or minimally invasive procedure to correct structural defects in the heart valves. As used in this disclosure, “stent placement” is the insertion of a small, expandable tube, or stent, into a narrowed or blocked blood vessel to restore and maintain blood flow.

With continued reference to FIG. 1, the ultrasonic imaging may be performed using different modalities based on the specific procedural requirements. For imaging within the heart, an intracardiac echocardiogram (ICE) may be employed to provide real-time, high-resolution images of intracardiac structures. For imaging within the esophagus, a transesophageal echocardiogram (TEE) may offer a visualization of the heart and its valves. Additionally and or alternatively, imaging outside the chest, such as transthoracic echocardiogram (TTE) or point of care ultrasound (POCUS), may be used for non-invasive assessments. As used in this disclosure, an “intracardiac echocardiogram (ICE)” is a diagnostic imaging technique that uses a catheter 108 equipped with ultrasound transducers to produce high-resolution, real-time images of intracardiac structures and adjacent anatomy during interventional procedures. As used in this disclosure, a “transesophageal echocardiogram (TEE)” is an imaging method where an ultrasound probe is inserted into the esophagus to capture detailed images of the heart and its surrounding structures. As used in this disclosure, “point of care ultrasound (POCUS)” is a portable, bedside ultrasound imaging technique used by clinicians for immediate assessment of a patient's condition. The integration of these ultrasonic modalities within the robotic framework 106 allows for dynamic and flexible imaging capabilities. The produced 3D models 142 enhance procedural planning and execution by providing detailed anatomical visualizations. These models help in guiding interventions, verifying device placements, and assessing post-procedural outcomes with precision, improving patient safety and procedural efficacy.

With continued reference to FIG. 1, as used in this disclosure, a “catheter” is a medical device designed to be inserted into a body to facilitate diagnostic, therapeutic, or interventional procedures. Catheters may include biocompatible materials such as polyurethane, silicone, or polyethylene and may include various sizes, lengths, and configurations. In a non-limiting example, a catheter 108 may be used to introduce imaging devices, such as ultrasonic transducers, into blood vessels to capture internal anatomical information or to deliver medical treatments, such as medications or interventional tools, to targeted regions. The catheter 108 may include features such as sensors, guide wires, or lumens to support its specific medical application. A catheter 108 can be inserted into a coronary artery with an empty balloon and a stent attached to the end. For the purposes of this disclosure, a “stent” is a medical device designed to be implanted within a bodily lumen to restore or maintain openness by providing structural support. Stents may include tubular in shape and may include biocompatible materials such as stainless steel, cobalt-chromium alloys, nitinol (a shape-memory alloy), or biodegradable polymers.

With continued reference to FIG. 1, the apparatus 100 may include different hardware for specific measurements. In some embodiments, hardware may be transducers, sensors, and actuators. For the purposes of this disclosure, a “transducer” is a device used to transform one kind of energy into another. When a transducer converts a quantity of energy to an electrical voltage or an electrical current it may be called a sensor. A measurable quantity of energy may include sound pressure, optical intensity, magnetic field intensity, thermal pressure, etc. When a transducer converts an electrical signal into another form of energy such as sound, light, mechanical movement, it is called an actuator. It should be noted that sound is incidentally a pressure field. Actuators allow the use of feedback at the source of the measurements.

With continued reference to FIG. 1, a sensor may be considered as a component or with a collection of electronics such as amplifiers, decoders, filters, computer devices and the apparatus 100. For the purposes of this disclosure an “instrument” is a sensor bundled with its associated electronics. However, in some embodiments, sensors may be further integrated with the apparatus 100. For example, without limitation, the robotic framework 106 may include one or more sensors.

With continued reference to FIG. 1, a sensor integrated with the apparatus 100 may be linear so that response y to a stimulus x is in the form: y(x)=Ax, 0≤x≤xₘₐₓ, A>0. It should be noted, there is a presumption that the stimulus to be positive. A is the sensitivity of the transducer gain, or the gain of the sensor. The gain is presumed to be positive for which the linear model satisfies the definition of linearity: y(x+z)=A(x+z)=y(x)+y(z). It should be noted that this example is an idealized form of a sensor and may extend beyond the linearity constraints which may include time dependency, memory, and its output keeping track of input. A more generalized sensor may include the steady state transfer function of the sensor. For this case, the sensitivity can be defined as the derivative of the output with respect to the input:

$$S = \frac{\partial y}{\partial x} .$$

In this example, the sensor exhibits sensitivities to other operating parameters 148 (i.e. supply voltage) or temperature. For the purposes of this disclosure, “sensitivity” is the ratio of output to input. This can include electrical output and signal input or an input transducer. It can also include physical output to an electrical input, or an output transducer. Sensitivity can also be used in its usual electrical meaning. In this it would refer to a percent change of a property of a device because of a percent change in a parameter. In some embodiments this would be a percent change in gain as a result of percent change in ambient temperature. This type of sensitivity may be referred to as the Gain of a sensor.

Still referring to FIG. 1, the apparatus 100 with integrated sensors may not respond to arbitrarily small signals. The apparatus 100 may respond to signals within a specified range from zero to a sensor threshold which does not cause the output of the sensor to change. The existence of a threshold relates to the nonlinear behavior of the device and the noise. The apparatus 100 with an integrated sensor may fail to respond to stimuli which are arbitrarily large as well. In this case, the apparatus 100 integrated with a sensor may have a max range. The full range of the apparatus 100 integrated with a sensor may be limited by compression or clipping. Compression and clipping are results of nonlinearity and thus may include the apparatus 100 as a nonlinearity device.

Still referring to FIG. 1, referring to the linear equation above assuming a linear sensor is improved with the addition of a constant: y(x)=b₀+Ax. It should be noted that the equation is not linear even though it is described as a first order polynomial. The constant is called a zero offset and can be defined in two ways: a sensor reading when the input is zero, or the value of the stimulus required to make the output zero. The zero offset is corrected by subtracting b₀ from y and recovering the linear description of a sensor: y′(x)=y(x)−b₀=Ax.

With continued reference to FIG. 1, the apparatus 100 may include very fast measurements where it can internally store energy. The apparatus 100 output may depend on previous measurements the integrated sensors make. It should be noted that the sensor may exhibit memory. The time dependence of a sensor can be linear if the response is described by a linear differential equation:

$${\sum }_{n = 0}^{N} {A}_{n} \frac{{\partial }^{n} y}{\partial {t}^{n}} = {\sum }_{k = 0}^{K} {B}_{k} \frac{{\partial }^{k} x}{\partial {t}^{k}} .$$

Taking the Laplace transform of this equation:

$$y ( s , X ) = ( \frac{{\sum }_{k = 0}^{K} {B}_{k} {S}^{k}}{{\sum }_{n = 0}^{N} {A}_{n} {S}^{n}} ) x = H ( s ) X ( s ) ,$$

which is in Laplace transform space and the sensor response is still linear in stimulus x. The response of a sensor with a transfer function H(s) at time tis the convolution integral between the history of the stimulus x and the inverse Laplace transform h(t) of H(s):

$$y ( t ) = {\int }_{0}^{∞} h ( τ ) x ( t - τ ) d τ .$$

The apparatus 100 may behave like a low pass filter, wherein there is a delayed response to their input. There is a limit to the maximum stimulus frequency that can be detected. The maximum frequency a sensor can interpret is approximately the inverse of its response time.

With continued reference to FIG. 1, in an embodiment, the at least a transducer 110 may convert one form of energy into another for the purpose of sensing, measurement, or signal generation. In a non-limiting example, the at least a transducer 110 may be configured to convert electrical signals into ultrasonic waves and vice versa, enabling it to transmit and receive acoustic energy for imaging or therapeutic purposes. The transducer 110 may include piezoelectric elements, which generate ultrasonic signals 112 when electrically stimulated, and detect returning echoes to provide data for creating detailed images of internal structures. In an embodiment, the transducer 110 may include an ultrasonic transducer. As used in this disclosure, an “ultrasonic transducer” is a device that converts electrical energy into ultrasonic waves and ultrasonic waves back into electrical energy for purposes such as imaging, measurement, or therapeutic applications. In a non-limiting example, an ultrasonic transducer may utilize piezoelectric elements that vibrate when an electrical signal is applied, generating high-frequency sound waves that propagate through a medium. The transducer 110 may then detect echoes of these waves reflected from structures within the medium and convert them into electrical signals, which can be processed to generate images or provide diagnostic information. In an embodiment, the ultrasonic transducer may create ultrasound images by generating and detecting ultrasonic waves. As used in this disclosure, an “ultrasound image” is a visual representation generated by reflection of high-frequency sound waves off internal body structures. In a non-limiting example, ultrasound image may include visual representation of a heart examined through esophagus. As a non-limiting example, ultrasound image may include distance between sensor and surrounding tissue or organs. In some cases, ultrasound transducer may detect ultrasound image in a plurality of angles. In a non-limiting example, ultrasound image may include a plurality of distances between sensor and a heart in different angles. For example, and without limitation, when ultrasound transducer moves around within an esophagus, ultrasound transducer receives a plurality of distances between ultrasound transducer and organ and generate ultrasound image using the plurality of distances. As another non-limiting example, ultrasound transducer may include signal strength or amplitude of ultrasonic signal 112 emitted and received by ultrasound sensor, images within an organ, or the like. As another non-limiting example, ultrasound transducer may include ambient temperature, humidity, atmospheric pressure, or the like. In some embodiments, ultrasound transducer may be stored in a database. In some embodiments, ultrasound image may be retrieved from database. In some embodiments, user may manually input ultrasound image. In some embodiments, ultrasound image may be received from remote device. As a non-limiting example, processor may receive ultrasound transducer from a computing device or processor incorporated with ultrasound transducer or TEE apparatus.

With continued reference to FIG. 1, in a non-limiting example, the transducer 110 may incorporate capacitive micromachined ultrasonic transducer (CM UT) technology, which utilizes microscopic membranes to generate and receive ultrasonic waves. This configuration may allow the transducer 110 to operate at higher frequencies, enabling enhanced resolution for imaging applications such as intravascular ultrasound (IVUS) or other minimally invasive procedures. The transducer 110 may be operatively coupled to a catheter 108, permitting real-time imaging of internal structures such as blood vessels, cardiac tissues, or other anatomical regions. In a non-limiting example, the robotic framework 106 may include a multi-axis robotic arm 120 operatively connected to a catheter 108 coupled to an ultrasonic transducer. Continuing, the transducer 110 may be configured to emit and receive a plurality of ultrasonic signals 112 to capture detailed imaging data of a target region 118, as discussed in more detail below, within the body. For instance, without limitation, the robotic framework 106 may control the precise positioning of the catheter 108 to navigate through blood vessels and optimize the orientation of the transducer 110 for improved imaging quality. Without limitation, this configuration may enable real-time acquisition of three-dimensional (3D) models of anatomical structures, such as vascular walls or cardiac tissues, facilitating diagnostic or therapeutic procedures, as discussed more herein. In another non-limiting example, the robotic framework 106 may incorporate feedback from a localization system 114, as discussed below, to dynamically adjust the position of the catheter 108 and the transducer 110 in response to changes in the target region 118. Continuing, the transducer 110, configured to receive a plurality of ultrasonic signals 112, may collect data that is processed by a computing device to refine imaging outputs or guide the movements of the robotic framework 106. For example, the apparatus may adaptively adjust the trajectory of the catheter 108 to maintain continuous imaging of a moving target, such as a beating heart or blood flow, ensuring accurate data collection and enhanced procedural outcomes.

Still referring to FIG. 1, the apparatus includes at least a localization system 114 configured to detect at least a position signal 116 as a function of a location of the catheter 108. As used in this disclosure, a “localization system” is an apparatus that detect and determines the position of a catheter within a body or environment. These signals are a function of at least a catheter 108 location, enabling precise tracking and navigation during medical procedures. In a non-limiting example, the purpose of at least a localization system 114 is to enhance the safety and efficacy of catheter-based interventions by providing critical spatial information. As used in this disclosure, a “position signal” is a signal generated by localization system 114 to determine the location of a catheter 108 within the body. In a non-limiting example, the position signal 116 may be derived from ultrasonic feedback, where the localization system 114 uses an array of transducers to emit ultrasonic waves and detect their reflections from the catheter 108. The resulting position signals 116 may indicate the spatial coordinates of the catheter 108 within the body and can be processed to adjust its trajectory dynamically. In a non-limiting example, position signals 116 may be represented as Cartesian coordinates (x, y, z) that define the location of the catheter 108 within a three-dimensional (3D) space. For instance, the localization system 114 may generate position signals 116 such as (12.5, 8.3, 4.7), indicating the exact spatial position of the catheter 108 relative to a defined origin point. Continuing, these coordinates may be continuously updated in real time to reflect the movement of the catheter 108, allowing precise tracking and guidance during a medical procedure. In another non-limiting example, position signals 116 may include additional data such as orientation or angular displacement. For instance, the position signals 116 may take the form of a six-dimensional representation (x, y, z, roll, pitch, yaw), where the first three dimensions define the location and the latter three define the orientation of the catheter 108. Continuing, this may enable the localization system 114 to provide more detailed guidance, particularly for tasks requiring precise alignment, such as positioning an ultrasonic transducer at a specific angle relative to a target region 118. In another non-limiting example, the position signals 116 may be expressed as polar coordinates (r, θ, φ), where “r” represents the radial distance from a reference point, “θ” the azimuthal angle, and “φ” the polar angle. Such a representation may be particularly useful in applications involving circular or spherical navigation paths, such as guiding a catheter 108 around the walls of a heart chamber or within a curved blood vessel. Continuing, these position signals 116 may then be converted into actionable movement instructions for the robotic framework 106 to control the trajectory of the catheter 108 effectively.

With continued reference to FIG. 1, in a non-limiting example, localization system 114 may be consistent with one or more aspects of the localization system described in attorney docket number 1518-160USU1, U.S. patent application Ser. No. 18/764,853, filed on Jul. 5, 2024, titled “SYSTEM AND METHOD FOR LOCATING A MEDICAL DEVICE USING AN ELECTRICAL FIELD CREATION,” which is incorporated by reference herein in its entirety.

With continued reference to FIG. 1, as used in this disclosure, an “ultrasound-based localization system” is a method used to determine the position and movement of objects within the body by employing high-frequency sound waves. The ultrasound-based localization system may involve the use of an ultrasound transducer that emits sound waves, which then reflect off internal structures and are captured by transducer 110 or other sensors. Continuing, the reflected sound waves are processed to create real-time images or data points that represent the location and motion of the tracked object, such as a catheter 108 or other medical instruments. The ultrasound-based localization system may be particularly useful in medical procedures because it provides real-time, non-invasive visualization of internal body structures. The ultrasound-based localization system may allow clinicians to guide instruments accurately within the body, enhancing the precision and safety of procedures like catheter ablation, biopsies, or other interventions. This technology is often integrated with other systems to provide comprehensive spatial and functional mapping of the area being treated. For example, at least a localization system 114 may utilize ultrasound technology, where an array of ultrasound transducers is positioned around the patient. At least a catheter 108 may be fitted with miniature ultrasound receivers, detects the emitted ultrasound waves. At least a localization system 114 may calculate at least a catheter 108 position based on the time it takes for the ultrasound waves to reach the receivers, allowing for precise localization of at least a catheter tip during a procedure.

With continued reference to FIG. 1, as used in this disclosure, an “ultrasonic signal” is a form of acoustic energy that propagates as sound waves with frequencies above the range of human hearing. In an embodiment, the plurality of ultrasonic signals 112 may include frequencies greater than 20 kilohertz (kHz). In a non-limiting example, the plurality of ultrasonic signals 112 may be generated by the transducer 110 for applications such as imaging or therapeutic procedures. The plurality of ultrasonic signals 112 may travel through a medium, such as biological tissue, and reflect off structures within the medium to provide information about their location, composition, or movement. These reflected signals may then be captured and analyzed to generate detailed visualizations or to perform specific medical or industrial tasks.

Still referring to FIG. 1, the at least a processor receives a target region 118. As used in this disclosure, a “target region” is an area or location within a subject or an environment 150 that is designated for imaging or analysis. In a non-limiting example, the target region 118 may include an anatomical structure such as a blood vessel, an organ, or a lesion within the body of a subject that is intended for diagnostic imaging, therapeutic treatment, or procedural navigation. The target region 118 may be identified based on input data, such as preoperative imaging scans, and may guide the operation of devices such as the robotic framework 106 to achieve precise and effective outcomes.

With continued reference to FIG. 1, the robotic framework 106 further may include a robotic arm 120 coupled to an end effector 122, wherein the robotic arm 120 is configured to provide multi-axis movement relative to the target region 118. As used in this disclosure, a “robotic arm” is a mechanical device designed to mimic at least a portion of the motion and functionality of a human arm. In an embodiment, the robotic arm 120 may include multiple segments connected by joints that enable multi-axis movement. In a non-limiting example, the robotic arm 120 may include actuators, such as motors or hydraulic systems, to control its motion and encoders to provide real-time feedback on its position and orientation. The robotic arm 120 may be configured to perform tasks such as manipulating tools, positioning instruments, or navigating catheters with precision, often under the control of a computing device or feedback system.

With continued reference to FIG. 1, in an embodiment, the robotic framework 106 may include a system of drive wheels configured to manipulate the catheter 108 or guidewire independently of the movement of the robotic arm 120. Continuing, the drive wheels may be positioned within a control module and designed to advance, retract, or rotate the catheter 108 or guidewire with high precision. In this configuration, without limitation, the robotic arm 120 may remain stationary or focus on broader positioning tasks, while the drive wheels provide fine control over the movements of the apparatus 100. The use of drive wheels may enable more compact and streamlined robotic designs by localizing actuation mechanisms near the catheter 108 or guidewire interface. Furthermore, the apparatus 100 may include sensors and feedback mechanisms to ensure that the drive wheels operate with the accuracy and safety required for delicate medical procedures.

With continued reference to FIG. 1, the robotic framework 106 may include various features and functionalities suitable for applications. Without limitation, the robotic framework 106 may include similar features as robotic-assisted percutaneous coronary intervention (PCI). As used in this disclosure, a “robotic-assisted percutaneous coronary intervention” is a medical procedure in which robotic systems are employed to enhance the precision and control of catheter-based interventions for treating coronary artery disease. The procedure involves the use of robotic technologies to assist clinicians in navigating and deploying interventional devices, such as guidewires and stents, through small incisions to reach and treat blockages in the coronary arteries. The term includes systems that provide remote operation capabilities, improve ergonomic efficiency for the operator, and reduce exposure to radiation while ensuring high accuracy during the intervention. In a non-limiting example, the robotic framework 106 may include a robotic control console that allows the operator to manipulate interventional devices with fine precision through a joystick or touchscreen interface, and may be augmented by haptic feedback for enhanced tactile sensation. The robotic framework 106 may also incorporate a robotic arm with drive mechanisms capable of precise handling of guidewires, catheters, and other devices, enabling controlled insertion, rotation, and retraction.

Additionally and or alternatively, the robotic framework 106 may integrate real-time imaging modalities, such as fluoroscopy or intravascular ultrasound (IVUS), to provide detailed visual feedback for guiding device navigation and deployment. The robotic framework 106 may further support remote operation capabilities, allowing the operator to perform procedures from another location that may improve ergonomic efficiency. Automated safety features, such as force sensing and collision avoidance mechanisms, may be included in the apparatus 100 to enhance patient safety by preventing excessive pressure on vessel walls or unintended device movements. In a non-limiting example, force sensing mechanisms in the robotic framework 106 may include strain gauge sensors integrated into the robotic arm 120 or catheter drive system. As used in this disclosure, a “strain gauge sensor” is a device that measures the amount of strain in an object when a force is applied. The strain gauge sensor may include a conductive material, such as a metal or semiconductor, whose electrical resistance changes in proportion to the strain it experiences. The strain gauge sensor can provide precise information about the magnitude and direction of the applied force by measuring the change in resistance. The strain gauge may include various configurations, such as foil, wire, or thin-film strain gauges, and may be integrated into the robotic framework 106 to monitor forces acting on medical instruments or other components. Continuing, these sensors may measure the force exerted on the guidewire or catheter and provide real-time feedback to the operator, preventing excessive force that could risk damaging the arterial walls. As used in this disclosure, a “catheter drive system” is a mechanical or electromechanical subsystem designed to control the movement of a catheter during medical procedures. Without limitation, the catheter drive system may include motors, gears, rollers, or other actuating elements that enable precise control of the insertion, retraction, rotation, and stabilization of the catheter 108. The catheter drive system may further include systems that incorporate sensors, feedback mechanisms, and software algorithms to ensure accurate and safe catheter navigation within the body, such as through arteries during invasive procedures. Another non-limiting example may include optical force sensors that use fiber optics to detect minute changes in pressure and deliver precise measurements for enhanced control during device manipulation. For collision avoidance mechanisms, without limitation, the robotic framework may include proximity sensors, such as ultrasonic or infrared sensors, that detect the relative position of the catheter 108 or guidewire to surrounding anatomical structures. These sensors may trigger automatic adjustments or alarms if the device approaches vessel walls or sensitive areas. Continuing the previous non-limiting example, the apparatus 100 may utilize advanced algorithms that analyze imaging data in real-time to predict and prevent collisions by automatically adjusting the trajectory of the robotic arm 120 or catheter 108 to maintain safe distances from critical structures.

With continued reference to FIG. 1, the robotic framework 106 may leverage advanced software, including machine learning algorithms, to assist with decision-making, optimize device placement, and predict potential complications. Without limitation, the apparatus 100 may feature a user-friendly interface for seamless operation and sterile field adaptability to ensure compliance with infection control protocols, while additional features like voice-activated commands may further improve workflow efficiency. In another non-limiting example the robotic framework 106 may include precise positioning mechanisms, high-resolution imaging integration, and real-time feedback systems to enhance procedural accuracy. Without limitation, the robotic framework 106 may be configured to support remote operation, allowing the operator to perform interventions from another location. For instance, without limitation, the operator may be in a shielded location potentially reducing radiation exposure. Additionally and or alternatively, the robotic framework may include advanced sensor technologies to monitor and adapt to dynamic changes during the procedure.

With continued reference to FIG. 1, as used in this disclosure, an “end effector” is a device or tool attached to the distal end of a device, designed to interact with the environment or perform specific tasks. In a non-limiting example, the end effector 122 may include components such as grippers for holding objects, needles for medical procedures, transducers for imaging applications, and the like. The end effector 122 may be interchangeable and customizable, enabling the robotic framework 106 to adapt to a variety of functions, such as surgical assistance, diagnostic imaging, or material handling.

With continued reference to FIG. 1, in a non-limiting example, the robotic arm 120 may support an end effector 122 equipped with an ultrasonic transducer for imaging applications. The robotic framework 106 may adjust the position and angle of the transducer 110 relative to the target region 118, such as a lesion in an organ or tissue, to capture comprehensive imaging data. Continuing, by leveraging its multi-axis movement capabilities, the robotic arm 120 may perform a controlled sweep across the target region 118, dynamically adapting to the anatomical contours and providing high-resolution three-dimensional (3D) imaging of the area of interest.

Still referring to FIG. 1, the at least a processor receives, from the localization system 114, the at least a position signal 116 of the catheter 108. The position signal 116 may provide precise spatial coordinates, such as (x, y, z), enabling the processor to identify the exact placement of the catheter 108 relative to a predefined target region 118. Continuing, this real-time data may allow the processor to dynamically adjust the movement of the catheter 108, ensuring accurate navigation through anatomical structures. The processor may further interpret the position signal 116 in conjunction with other inputs, such as feedback from an imaging system or preoperative scans, to optimize the trajectory of the catheter 108. For instance, if the position signal 116 indicates a deviation from the planned path, the processor may calculate corrective movements to realign the catheter 108. This capability may enhance the precision and safety of medical procedures by ensuring the catheter 108 remains on course during complex navigation tasks.

Still referring to FIG. 1, the at least a processor generates, using at least a reinforcement-learning model 124, a movement vector 126 as a function of the at least a position signal 116. As used in this disclosure, a “reinforcement-learning model” is a computational model that uses machine learning techniques to optimize decision-making or control tasks through interactions with an environment 150. In some embodiments, environment 150 may be virtual. Without limitation, the reinforcement-learning model 124 may learn by receiving feedback in the form of rewards or penalties based on the actions it takes and iteratively updates its parameters 148 to maximize cumulative rewards over time. In a non-limiting example, the reinforcement-learning model 124 may be employed to guide the movement of a robotic framework 106 by generating movement vectors 126 that optimize the trajectory of a catheter 108, based on positional feedback and a defined reward function 128, such as minimizing the deviation from a target path or maximizing the accuracy of imaging. The reinforcement-learning model 124 may be trained through simulations, real-world interactions, or a combination of both, and may incorporate additional learning approaches, such as supervised learning, to refine its performance.

With continued reference to FIG. 1, as used in this disclosure, a “reward function” is a mathematical or algorithmic construct used in a reinforcement-learning model 124 to quantify the desirability of an action or sequence of actions taken within an environment 150. The reward function 128 assigns numerical values, referred to as rewards or penalties, based on how well an action aligns with predefined objectives or desired outcomes. In a non-limiting example, a reward function 128 may evaluate the trajectory of a catheter 108 guided by a robotic framework 106, assigning positive rewards for actions that minimize the distance to a target region 118 or improve imaging accuracy, and assigning penalties for deviations or inefficient movements. The reward function 128 may serve as a critical component of the learning process, enabling the reinforcement-learning model 124 to iteratively optimize its behavior to achieve the best possible performance.

In a non-limiting example, a reward function 128 may be defined to guide the movement of the catheter 108 toward a target region 118 within a patient's body. The function could assign a positive reward proportional to the reduction in distance between the current position of the catheter 108 and the target region 118, such as R=−d(x, x_{target}), where d represents the Euclidean distance between the current position xₜ and the target x_{target}. If the catheter 108 moves closer to the target region 118, the reward increases, encouraging the reinforcement-learning model 124 to prioritize trajectories that minimize this distance. In another non-limiting example, the reward function 128 may evaluate imaging quality during the movement of the catheter 108. For instance, the function could assign a reward based on the signal-to-noise ratio (SNR) of ultrasonic signals 112 received by the transducer 110. A higher SNR, indicating clearer and more accurate imaging, might result in a larger positive reward, such asR=k*SNR, where k is a scaling factor. This encourages the system to adjust the position of the catheter 108 and orientation to maximize imaging clarity. In another non-limiting example, the reward function 128 may consider multiple objectives, such as minimizing trajectory deviation and maximizing imaging coverage. The function could take the form R=−αd(xₜ,x_{target})+βC, where d is the distance to the target, C represents imaging coverage (e.g., the percentage of the target region 118 scanned), and α and β are weight factors balancing the two objectives. This multi-objective reward function 128 enables the reinforcement-learning model 124 to find an optimal trade-off between precise navigation and comprehensive imaging.

With continued reference to FIG. 1, In a further non-limiting example, the reward function 128 could compare a 3D model generated from imaging data to a pre-existing CT scan of the target region 118, assigning a reward based on the minimization of error between the two models. For instance, the function might prioritize trajectories or movements that reduce discrepancies, such as R=−E, where E represents the error. Additionally and or alternatively, the reward function 128 may incorporate efficiency considerations, optimizing for the fewest number of imaging passes required as long as the error remains below a predefined threshold. In a non-limiting example, the reward function 128 may be designed to optimize efficiency by minimizing the number of imaging passes required to generate an accurate 3D model of the target region 118. The function could assign a negative penalty proportional to the number of passes made, such as R=−γP, where P represents the total number of passes and γ is a scaling factor. The reward function 128 may include a term that evaluates the error E between the generated 3D model and a reference CT scan of the target region. Continuing, if the error falls below a predefined threshold E_{threshold}, a positive reward R_{accuracy} may be assigned, encouraging the apparatus 100 to achieve the desired accuracy with fewer passes. For instance, without limitation, the reward function 128 may be illustrated by: R=R_{accuracy}−γP, where

$${R}_{accuracy} = \{ \begin{matrix}δ & if E \leq {E}_{threshold} \\ 0 & if E > {E}_{threshold}\end{matrix} ,$$

where δ is a positive reward for achieving the accuracy threshold.

With continued reference to FIG. 1, as used in this disclosure, a “movement vector” is a mathematical representation of direction and magnitude that specifies how an object should move within a space. In a non-limiting example, the movement vector 126 may include components such as spatial variables, such as x, y, z coordinates, temporal variables such as, speed or timing, and objective variables such as, distance to a target or alignment with a trajectory. Without limitation, the movement vector 126 may be generated by a computing device, often as a function of position signals 116 and other inputs, to guide the controlled movement of a device within an environment 150, such as navigating a catheter 108 through a patient's body or adjusting the position of an imaging transducer.

With continued reference to FIG. 1, the movement vector 126 may include one or more of a spatial variable 130 and a temporal variable 132. In an embodiment, the movement vector 126 may include an objective variable 134. As used in this disclosure, a “spatial variable” is a parameter that represents the position or orientation of an object within a defined space. In a non-limiting example, spatial variable 130 may include Cartesian coordinates (x,y,z) indicating the location of a catheter 108 in a three-dimensional environment or angular variables such as, pitch, roll, yaw, defining the orientation of an end effector 122. These variables enable precise tracking and manipulation of objects within the operational domain, such as guiding a catheter 108 toward a target region 118. As used in this disclosure, a “temporal variable” is a parameter that represents timing-related aspects of an action or movement within a system. In a non-limiting example, temporal variables 132 may include velocity such as, the speed of the movement of the catheter 108, acceleration, or the duration of a specific task. These variables can be used to ensure smooth and efficient operations, such as controlling the speed at which a catheter 108 approaches a target region 118 or adjusting timing to synchronize with dynamic events, such as cardiac motion. As used in this disclosure, an “objective variable” is a parameter that quantifies the performance or success of a system in achieving a specific goal. In a non-limiting example, objective variable 134 may include metrics such as the distance remaining to a target region 118, the quality of imaging data such as, signal-to-noise ratio, or the percentage of a target area successfully covered during scanning. These variables help guide decision-making processes by providing measurable outcomes that align with the system's overarching objectives, such as optimizing navigation or enhancing imaging clarity.

With continued reference to FIG. 1, the at least a reinforcement model may be trained using computed tomography training data 136. In an embodiment, the computed tomography data 136 may include historical computed tomography data 138 corresponding to historical movement vectors 140. As used in this disclosure, “computed tomography data” is data acquired from computed tomography (CT) imaging. Without limitation, CT imaging is a medical imaging technique that uses X-rays to create cross-sectional or three-dimensional (3D) images of internal structures within a subject. In a non-limiting example, CT data may include voxel-based representations of tissue density, with higher-density regions corresponding to structures such as bones, and lower-density regions representing softer tissues or organs. In a non-limiting example, CT data may be used to guide medical procedures, train machine learning models, or assist in planning and executing interventions by providing detailed anatomical information. As used in this disclosure, “historical movement vectors” are data representations that encapsulate the trajectory, speed, direction, or position changes of an object or entity over a defined past period. Historical movement vectors 140 may include information derived from tracking devices, sensors, or manual records and are utilized for analysis, prediction, or optimization purposes in applications such as navigation systems, logistics, sports performance analysis, or machine learning algorithms.

Still referring to FIG. 1, the at least a processor controls the robotic framework 106, as a function of the movement vector 126. Without limitation, the movement vector 126 may define the direction, magnitude, timing, and the like of the movement of the robotic framework 106, such as positioning a catheter 108 or adjusting the angle of an end effector 122. For instance, without limitation, the processor may calculate joint angles and actuator speeds required to move the robotic framework 106 along the trajectory specified by the movement vector 126. In a non-limiting example, the processor may continuously update the movement vector 126 of the robotic framework 106 based on real-time inputs, such as position signals 116 from a localization system 114 or feedback from sensors embedded in the robotic framework 106. Without limitation, the dynamic control may ensure that the robotic framework 106 responds effectively to changes in the environment 150, such as shifts in the target region 118 or patient movement. Additionally and or alternatively, the processor may use advanced algorithms, such as the reinforcement-learning model 124, to optimize the execution of the movement vector 126, improving precision and efficiency during complex tasks.

With continued reference to FIG. 1, the at least a processor 102 may receive the movement vector 126 from a planning module or an external input device and convert the movement vector 126 into control signals, which may then be transmitted to the robotic framework 106. As used in this disclosure, a “planning module” is a computational component configured to generate, refine, and optimize movement plans or trajectories for a robotic system based on input data and predefined objectives. The planning module may analyze factors such as the target region, environmental conditions, and system constraints to produce an output, such as a movement vector, that guides the robotic framework toward achieving a desired task. The term includes software and hardware implementations capable of performing operations like path planning, collision avoidance, and trajectory optimization. Additionally and or alternatively, the planning module may incorporate advanced algorithms, such as reinforcement learning or predictive modeling, to enhance its ability to adapt to dynamic conditions and improve the precision and efficiency of the robotic system's actions. Without limitation, the control signals may include specific instructions for motor actuation, such as torque commands, velocity commands, or positional adjustments, tailored to the actuators and joints of the robotic framework 106. In a non-limiting example, the control signals may be derived using inverse kinematics algorithms, ensuring that the robotic framework 106 follows the desired trajectory while maintaining appropriate alignment and stability. In another non-limiting example, the processor 102 may continuously update the movement vector 126 of the robotic framework 106 based on real-time inputs, such as position signals 116 from a localization system 114 or feedback from sensors embedded in the robotic framework 106. These sensors may provide information on joint positions, forces, or environmental interactions, allowing the processor 102 to dynamically refine the control signals and respond to any deviations from the planned trajectory. Without limitation, the dynamic control may ensure that the robotic framework 106 responds effectively to changes in the environment 150, such as shifts in the target region 118 or patient movement. Additionally and/or alternatively, the processor 102 may use advanced algorithms, such as the reinforcement-learning model 124, to optimize the execution of the movement vector 126, improving precision and efficiency during complex tasks.

Still referring to FIG. 1, the at least a processor generates, as a function of the plurality of ultrasonic signals 112, a three-dimensional (3D) model of the target region 118. As used in this disclosure, a “three-dimensional (3D) model” is a digital representation of an object, structure, or region that includes data about its geometry in three spatial dimensions. In a non-limiting example, a 3D model 142 may be generated from imaging data, such as ultrasonic signals 112, computed tomography scans, or magnetic resonance imaging (M RI) data, to provide a detailed visualization 146 of internal anatomical structures. The 3D model 142 may be used for various purposes, including diagnostic analysis, surgical planning, or real-time navigation during medical procedures, offering enhanced spatial understanding and precision. In a non-limiting example, the plurality of ultrasonic signals 112 may be used to capture real-time imaging of the internal structures of the heart, such as the chambers, valves, blood flow dynamics, and the like. The ultrasonic transducer may emit high-frequency sound waves that propagate through the chest wall and reflect off cardiac tissues. These reflected ultrasonic signals 112 may be processed by a computing device to generate detailed two-dimensional (2D) or three-dimensional (3D) images of the heart, allowing clinicians to monitor the heart's function and detect abnormalities, such as valve defects or arrhythmias.

With continued reference to FIG. 1, in a non-limiting example, the 3D model 142 may be consistent with one or more aspects of the 3D model described in attorney docket number 1518-103USU1, U.S. patent application Ser. No. 18/376,688, filed on Oct. 4, 2023, titled “APPARATUS AND METHODS FOR GENERATING A THREE-DIMENSIONAL (3D) MODEL OF CARDIAC ANATOMY VIA MACHINE-LEARNING,” which is incorporated by reference herein in its entirety.

With continued reference to FIG. 1, in a non-limiting example, the 3D model 142 may be consistent with one or more aspects of the 3D model described in attorney docket number 1518-103USC1, U.S. patent application Ser. No. 18/750,411, filed on Jun. 21, 2024, titled “APPARATUS AND METHODS FOR GENERATING A THREE-DIMENSIONAL (3D) MODEL OF CARDIAC ANATOMY VIA MACHINE-LEARNING,” which is incorporated by reference herein in its entirety.

With continued reference to FIG. 1, in a non-limiting example, the 3D model 142 may be consistent with one or more aspects of the 3D model described in attorney docket number 1518-103USC2, U.S. patent application Ser. No. 18/818,034, filed on Aug. 28, 2024, titled “APPARATUS AND METHODS FOR GENERATING A THREE-DIMENSIONAL (3D) MODEL OF AN ANATOMICAL OBJECT VIA MACHINE-LEARNING,” which is incorporated by reference herein in its entirety.

With continued reference to FIG. 1, in a non-limiting example, the 3D model 142 may be consistent with one or more aspects of the 3D model described in attorney docket number 1518-104USU1, U.S. patent application Ser. No. 18/389,513, filed on Nov. 14, 2023, titled “APPARATUS AND METHODS FOR SYNTHESIZING MEDICAL IMAGES,” which is incorporated by reference herein in its entirety.

With continued reference to FIG. 1, in a non-limiting example, the 3D model 142 may be consistent with one or more aspects of the 3D model described in attorney docket number 1518-104USC1, U.S. patent application Ser. No. 18/817,870, filed on Aug. 28, 2024, titled “APPARATUS AND METHODS FOR SYNTHESIZING MEDICAL IMAGES,” which is incorporated by reference herein in its entirety.

With continued reference to FIG. 1, in a non-limiting example, the 3D model 142 may be consistent with one or more aspects of the 3D model described in attorney docket number 1518-105USU1, U.S. patent application Ser. No. 18/426,604, filed on Jan. 30, 2024, titled “APPARATUS AND METHODS FOR GENERATING A THREE-DIMENSIONAL (3D) MODEL OF CARDIAC ANATOMY BASED ON MODEL UNCERTAINTY,” which is incorporated by reference herein in its entirety.

With continued reference to FIG. 1, in a non-limiting example, the 3D model 142 may be consistent with one or more aspects of the 3D model described in attorney docket number 1518-105USC1, U.S. patent application Ser. No. 18/818,152, filed on Aug. 28, 2024, titled “APPARATUS AND METHOD FOR GENERATING A THREE-DIMENSIONAL (3D) MODEL OF PATIENTS ORGAN,” which is incorporated by reference herein in its entirety.

With continued reference to FIG. 1, in a non-limiting example, the 3D model 142 may be consistent with one or more aspects of the 3D model described in attorney docket number 1518-105USC2, U.S. patent application Ser. No. 19/001,840, filed on Dec. 26, 2024, titled “APPARATUS AND METHOD FOR GENERATING A THREE-DIMENSIONAL (3D) MODEL OF CARDIAC ANATOMY BASED ON MODEL UNCERTAINTY,” which is incorporated by reference herein in its entirety.

With continued reference to FIG. 1, in a non-limiting example, the 3D model 142 may be consistent with one or more aspects of the 3D model described in attorney docket number 1518-110USU1, U.S. patent application Ser. No. 18/395,087, filed on Dec. 22, 2023, titled “APPARATUS AND METHODS FOR GENERATING A THREE-DIMENSIONAL (3D) MODEL OF CARDIAC ANATOMY WITH AN OVERLAY,” which is incorporated by reference herein in its entirety.

With continued reference to FIG. 1, in a non-limiting example, the 3D model 142 may be consistent with one or more aspects of the 3D model described in attorney docket number 1518-110USC1, U.S. patent application Ser. No. 18/818,311, filed on Aug. 28, 2024, titled “APPARATUS AND METHOD FOR GENERATING A THREE-DIMENSIONAL (3D) MODEL WITH AN OVERLAY,” which is incorporated by reference herein in its entirety.

With continued reference to FIG. 1, the at least a processor 102 may be further configured to generate a visualization 146 based on the 3D model 142 of the target region 118, and display the visualization 146 using a display device 144. As used in this disclosure, a “display device” refers to an electronic device that visually presents information to the entity. In some cases, display device 144 may be configured to project or show visual content generated by computers, video devices, or other electronic mechanisms. In some cases, display device 144 may include a liquid crystal display (LCD), a cathode ray tube (CRT), a plasma display, a light emitting diode (LED) display, and any combinations thereof. In a non-limiting example, one or more display devices 144 may vary in size, resolution, technology, and functionality. Display device 144 may be able to show any data elements and/or visual elements as listed above in various formats such as, textural, graphical, video among others, in either monochrome or color. Display device 144 may include, but is not limited to, a smartphone, tablet, laptop, monitor, tablet, and the like. Display device 144 may include a separate device that includes a transparent screen configured to display computer generated images and/or information. In some cases, display device 144 may be configured to present a graphical user interface (GUI) to a user, wherein a user may interact with a GUI. In some cases, a user may view a GUI through display. Additionally, or alternatively, processor be connected to display device 144. In one or more embodiments, transmitting the visualization 146 may include displaying the visualization 146 at display device 144 using a visual interface. As used in this disclosure, a “visual interface” is a digital display that presents information, options, interactive elements to users in an intuitive and visually appealing manner. In some embodiments, visual interface may include at least an interface element. As used in this disclosure, “at least an interface element” is a portion of visual interface. In a non-limiting example, at least an interface element may include, without limitation, a button, a link, a checkbox, a text entry box and/or window, a drop-down list, a slider, or any other interface element that may occur to a person skilled in the art upon reviewing the entirety of this disclosure. In some embodiments, at least an interface element may include an event handler. An “event handler,” as used in this disclosure, is a module, data structure, function, and/or routine that performs an action in response to an event. For instance, and without limitation, an event handler may record data corresponding to user selections of previously populated fields such as drop-down lists and/or text auto-complete and/or default entries, data corresponding to user selections of checkboxes, radio buttons, or the like, potentially along with automatically entered data triggered by such selections, user entry of textual data using a keyboard, touchscreen, speech-to-text program, or the like. Event handler may generate prompts for further information, may compare data to validation rules such as requirements that the data in question be entered within certain numerical ranges, and/or may modify data and/or generate warnings to a user in response to such requirements.

With continued reference to FIG. 1, the at least a reinforcement-learning model 124 is trained as a function of iterative updates to parameters 148 through interactions with an environment 150. As used in this disclosure, “parameters” are adjustable variables or coefficients within a reinforcement-learning model 124 that define its behavior and influence its decision-making process. In a non-limiting example, parameters 148 may include weights and biases in neural networks, which are updated during the training process to optimize the performance of the reinforcement-learning model 124. These parameters 148 are iteratively refined through interactions with an environment 150, where the reinforcement-learning model 124 may evaluate outcomes, receives feedback in the form of rewards or penalties, and adjusts its parameters 148 to improve future decisions. This iterative process may enable the reinforcement-learning model 124 to adapt and enhance its ability to perform specific tasks, such as generating movement vectors 126 for controlling the robotic framework 106. As used in this disclosure, an “environment” is the external system or context in which a reinforcement-learning model 124 operates, interacts, and learns. The environment 150 provides inputs to the reinforcement-learning model 124, such as sensory data or feedback, and responds to the actions of the reinforcement-learning model 124, allowing iterative adjustments to improve performance. In a non-limiting example, the environment 150 may represent a simulated or real-world scenario, such as the robotic framework 106 navigating the catheter 108 through a patient's anatomy. The environment 150 may include factors such as spatial constraints, dynamic obstacles, and predefined goals, which collectively influence the decision-making and learning process of the reinforcement-learning model 124. In a non-limiting example, the environment 150 may represent a simulated or real-world scenario. For example, without limitation, the robotic framework 106 may navigate the catheter 108 through a subject's anatomy or a simulated environment. The environment 150 may include factors such as spatial constraints, dynamic obstacles, and predefined goals, which collectively influence the decision-making and learning process of the reinforcement-learning model 124. Additionally and/or alternatively, the environment 150 may include simulated ultrasonic sweeps designed to emulate the imaging signals 112 generated by the transducer 110. These simulated sweeps may provide realistic feedback for the reinforcement-learning model 124 during training, allowing the model to refine its ability to optimize imaging quality and navigate complex anatomical structures in a controlled, risk-free setting.

With continued reference to FIG. 1, in a non-limiting example, the training process for the at least a reinforcement-learning model 124 may be consistent with one or more aspects of the model training described in attorney docket number 1518-203USU1, U.S. Pat. App. Ser. No. 19/188,841, filed on DATE Apr. 24, 2025, titled “SYSTEM AND METHOD FOR TRAJECTORY SWEEP SIMULATION,” which is incorporated by reference herein in its entirety.

With continued reference to FIG. 1, the at least a reinforcement-learning model 124 is trained using a supervised learning process 152, wherein the supervised learning process 152 may include training the at least a reinforcement-learning model 124 on labeled position data 154 representing historical catheter trajectory patterns 156. As used in this disclosure, a “supervised learning process” is a machine learning method in which a model is trained using labeled data, where each input is paired with a corresponding correct output or target. During the training process, the model iteratively adjusts its parameters 148 to minimize the error between its predicted outputs and the actual targets. In a non-limiting example, the supervised learning process 152 may involve training a reinforcement-learning model 124 using labeled position data 154 representing historical catheter trajectories. The labeled data may guide the model to learn patterns and optimal movements, improving its accuracy and reliability in navigating to a target region 118 or performing specific tasks. As used in this disclosure, “labeled position data” is information that includes specific positional information paired with corresponding labels or annotations that define its context, meaning, or desired outcome. In a non-limiting example, labeled position data 154 may consist of spatial coordinates of the trajectory of a catheter 108 within a three-dimensional environment, along with labels indicating the correct or optimal path for navigation. This data may be used in training machine learning models, such as a supervised learning process 152, to recognize and replicate effective movement patterns, ensuring precise navigation or alignment with a target region 118 during medical or robotic procedures. As used in this disclosure, “historical catheter trajectory patterns” refer to previously recorded data that represents the paths or movements a catheter has taken during past procedures. This data may include spatial coordinates and associated variables, such as velocity, angle, or timing, that describe how the catheter 108 navigated through specific anatomical regions. In a non-limiting example, historical catheter trajectory patterns 156 may be used to train a supervised or reinforcement-learning model 124 to identify optimal pathways, avoid obstacles, or improve precision in guiding the catheter 108 to a target region 118. These patterns may serve as a reference to refine the model's decision-making and enhance its ability to adapt to similar scenarios in future procedures.

With continued reference to FIG. 1, the movement vector 126 is dynamically adjusted in real-time based on feedback 158 from the localization system 114 and a comparison 160 of the position signal 116 to the target region 118. As used in this disclosure, “feedback” is information generated in response to an action within a system, which is subsequently used to evaluate or modify subsequent actions. In a non-limiting example, feedback 158 may include positional signal from the localization system 114 indicating the current location of a catheter 108 relative to a target region 118, or imaging quality metrics derived from the plurality of ultrasonic signals 112. Without limitation, the feedback 158 may be utilized by a computing device or a reinforcement-learning model 124 to dynamically adjust movements, optimize trajectories, or improve system performance during procedures such as medical navigation or imaging. In a non-limiting example, the movement vector 126 may be dynamically adjusted in real-time if feedback 158 from the localization system 114 indicates that the catheter 108 has deviated from its intended path. For instance, without limitation, if the position signal 116 indicates that the catheter 108 is slightly offset from the centerline of a blood vessel, the computing device may modify the movement vector 126 to correct the trajectory and re-align the catheter 108 with the target region 118, ensuring precise navigation. In another non-limiting example, the movement vector 126 may be updated in response to feedback 158 showing that the catheter 108 is approaching an obstacle, such as a bifurcation in a blood vessel. The ultrasonic transducer may detect the bifurcation by identifying distinct reflective surfaces that indicate vessel branching. This imaging data may be processed by the computing device, which can then adjust the movement vector 126 to guide the catheter 108 safely through the bifurcation while maintaining progress toward the target region 118. The localization system 114 may supplement this process by providing positional data to ensure the trajectory of the catheter 108 remains precise. In another non-limiting example, the movement vector 126 may be refined based on real-time changes in the target region 118 itself. For example, without limitation, the target region 118 may shift slightly due to the patient's heartbeat. Feedback 158 from the ultrasonic transducer may detect these changes by capturing variations in the reflected ultrasonic signals 112, providing detailed information about the new location of the target region 118. This data, combined with position signals 116 from the localization system 114, may enable the computing device to adjust the movement vector 126 dynamically, ensuring continuous alignment with the target region 118. This capability may improve the precision and reliability of the procedure, particularly in dynamic anatomical environments.

With continued reference to FIG. 1, the at least a reinforcement-learning model 124 is trained using a reinforcement learning process 162, wherein the reinforcement learning process 162 comprises optimizing a reward function 128 based on a comparison of the 3D model 142 to 3D medical imaging data 164. As used in this disclosure, a “reinforcement learning process” is a method in artificial intelligence where an entity interacts with an environment 150 to learn a sequence of actions that maximize a predefined reward signal. Without limitation, the reinforcement learning process 162 may include trial and error, where the entity receives feedback 158 in the form of rewards or penalties based on its actions, allowing it to refine its decision-making policy over time. The reinforcement learning process 162 may include components such as states, actions, policies, rewards, and value functions, enabling the entity to optimize its behavior for achieving a specific goal.

With continued reference to FIG. 1, in a non-limiting example, the reinforcement-learning model 124 may be trained using the reinforcement learning process 162 to guide the robotic framework 106 for transesophageal echocardiography (TEE) during cardiac imaging procedures. The reinforcement learning process 162 may involve the entity optimizing a reward function 128 that evaluates the alignment and clarity of the 3D models 142 generated from TEE imaging data compared to expert-labeled ground truth, or the 3D medical imaging data 164. The entity may receive rewards for achieving optimal catheter 108 positioning and imaging angles, enabling the robotic framework 106 to provide precise and consistent guidance during TEE procedures. In another non-limiting example, the reinforcement-learning model 124 may be trained to guide the robotic framework 106 in intracardiac echocardiography (ICE) during electrophysiology (EP) mapping procedures. For instance, the reinforcement learning process 162 may include the entity adjusting catheter 108 positions in real-time to optimize imaging of key cardiac structures while minimizing interference with the EP procedure. The reward function 128 may evaluate the quality of the 3D models 142 produced from ICE imaging data, penalizing suboptimal catheter 108 placement and incentivizing configurations that enhance visibility of anatomical landmarks, improving the safety and efficacy of the EP procedure. In another non-limiting example, the reinforcement-learning model 124 may be applied to optimize robotic guidance for catheter ablation in EP procedures. The reinforcement learning process 162 may guide the entity in maneuvering ICE catheters to provide clear 3D imaging of arrhythmogenic zones. The reward function 128 evaluates the accuracy of the 3D models 142 of the targeted regions compared to pre-procedural imaging and intraoperative annotations. As used in this disclosure, “ICE catheters” are specialized medical devices designed for intracardiac echocardiography (ICE). In an embodiment, ICE catheters may include a flexible catheter embedded with ultrasound transducers. Continuing, ICE catheters may be inserted into the heart via a vascular access point, such as the femoral vein, to provide real-time, high-resolution imaging of intracardiac structures and adjacent anatomy. ICE catheters may facilitate visualization 146 and guidance during various interventional cardiology procedures, including electrophysiological studies, catheter ablations, and structural heart interventions, enabling precise navigation and enhanced procedural outcomes. In another non-limiting example, a reinforcement-learning model 124 is trained to provide adaptive guidance for the robotic framework 106 during valve repair or replacement surgeries assisted by TEE imaging. The reinforcement learning process 162 may enable the entity to optimize catheter 108 manipulation and imaging angles based on real-time feedback 158 from the 3D model 142. The reward function 128 may compare the 3D TEE models to 3D medical imaging data 164, such as preoperative 3D imaging and intraoperative findings, rewarding configurations that improve visualization 146 of the surgical site, thus enhancing the precision and reliability of robotic-assisted interventions.

With continued reference to FIG. 1, the at least a reinforcement-learning model 124 is trained using a hybrid approach 166 combining reinforcement learning and supervised learning, wherein the reinforcement learning optimizes the reward function 128, and the supervised learning refines an adherence of the imitation model to known trajectory patterns. As used in this disclosure, a “hybrid approach” is a method that integrates reinforcement learning and supervised learning to leverage the complementary strengths of both techniques. Without limitation, reinforcement learning may focus on optimizing a reward function 128 through trial-and-error exploration, while supervised learning may train the at least a reinforcement-learning model 124 to align closely with known patterns or labeled data, enhancing the precision and reliability of the learning process. In a non-limiting example, the hybrid approach 166 may involve training at least a reinforcement-learning model 124 using reinforcement learning to optimize a reward function 128 for real-time adjustments during a procedure. Supervised learning may complement this process by refining the at least a reinforcement-learning model 124 to adhere to predefined trajectory patterns, such as those derived from expert-annotated 3D imaging datasets. Continuing, the hybrid approach 166 may enable the apparatus to benefit from the adaptive exploration capabilities of the reinforcement learning process 162 while ensuring the generated outputs remain consistent with known standards or clinical guidelines. Without limitation, a significant benefit of the hybrid approach 166 may include the balance it provides between adaptability and accuracy. The hybrid approach 166 may also ensure that real-time decisions made by the reinforcement learning process 162 are guided by the consistency and safety provided by supervised learning. For instance, in FIG. 1, the hybrid model may adapt to unforeseen challenges during a procedure while adhering to established trajectory patterns, ensuring safety and precision. This dual mechanism may make the hybrid approach 166 particularly suited for applications where both innovation and adherence to stringent operational constraints are critical.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

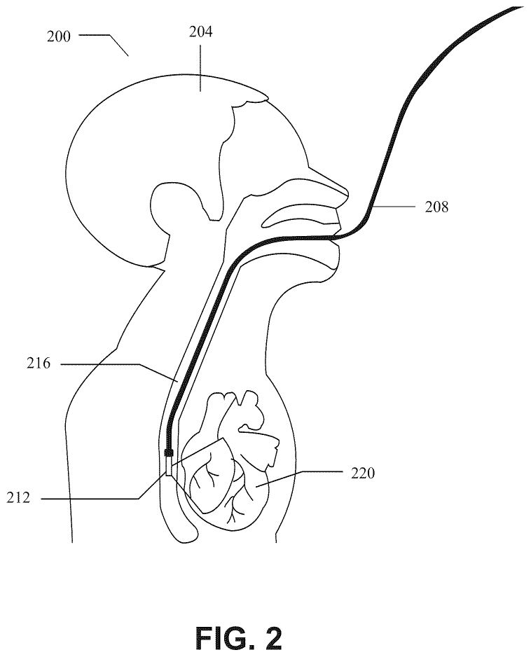

Now referring to FIG. 2, a schematic of an exemplary transesophageal echocardiogram (TEE) procedure 200 is shown. In some cases, TEE procedure 200 may be performed during another procedure for instance heart surgery. According to some embodiments, a patient 204 has an endoscope 208, with an ultrasonic transducer 212, inserted into his esophagus 216. As one's esophagus 216 is proximal one's heart 220, ultrasonic transducer 212 may generate echocardiograms.

Still referring to FIG. 2, in some embodiments, transesophageal echocardiography (TEE) may provide superior imaging quality than intracardiac echocardiography (ICE), as larger ultrasound transducers 212 may be placed within the esophagus 216 than within heart 220. In some cases, ultrasound transducers may be substantially miniaturized to fit within heart 220, as in ICE catheters. As esophagus 216 may be proximal to heart 220, TEE may provide a clear image of various heart structures without needing vascular access (as commonly required by ICE). Additionally, TEE may be performed without obstructing patient's 204 ribcage and intermediary tissues (as commonly required by transthoracic echocardiography [TTE]). In some cases, TEE images may also provide information associated with angle of acquisition. Angle of acquisition may be an angle of TEE probe with respect to esophagus 216 (e.g., esophageal axis).

Still referring to FIG. 2, in some embodiments, TEE echocardiogram data, including images showing heart structures and, in some cases, angle of acquisition, may be used as input to any machine learning process described in this application. For instance TEE echocardiogram data may be used to reconstruct 3D heart models. In some cases, TEE echocardiogram data may be input into a machine learning model that outputs a 3D heart model (e.g., 3D mesh model and/or statistical shape model).

Still referring to FIG. 2, in some embodiments, TEE may be a preferred imaging modality for structural heart interventions, such as without limitation left atrial appendage occlusion (LAOO), a stent, or a valve. In some cases, technology and improvements described in this disclosure permit creation and/or modification of a 3D heart mesh from TEE data to aid in planning implant size selection, as well as to guide implantation procedures. In some cases, virtual placement of a 3D model of a candidate implant (such as without limitation LAAO device) can be simulated on a 3D heart model generated by any method described in this disclosure. This novel and improved functionality may validate appropriate size and placement of implants within heart 220, as well as other organs within body of patient 204. For example, in the context of electrophysiology procedures, TEE procedure 200 can be used to create heart anatomical models that can be used as reference for electroanatomic mapping, and guidance of ablation catheters for atrial fibrillation treatment procedures (such as without limitation puncturing septum for Cardiovascular device placement).

Still referring to FIG. 2, in some embodiments, applications described with reference to TEE procedure 200 above can be extended for use with TTE and point of care ultrasound (POCUS). In some cases, both TTE and POCUS may acquire ultrasound images of chest/surface of patient 204. In some cases, TTE and POCUS data may be used as an input (and/or training data) for any machine learning process described in this disclosure. In some cases, TTE and POCUS may offer improved accessibility (with POCUS being portable/mobile as well) and non-invasive 3D heart modeling, often without anesthesia or sedation, compared to catheterized 3D heart modeling commonly performed today for electroanatomical mapping and ablation procedures.

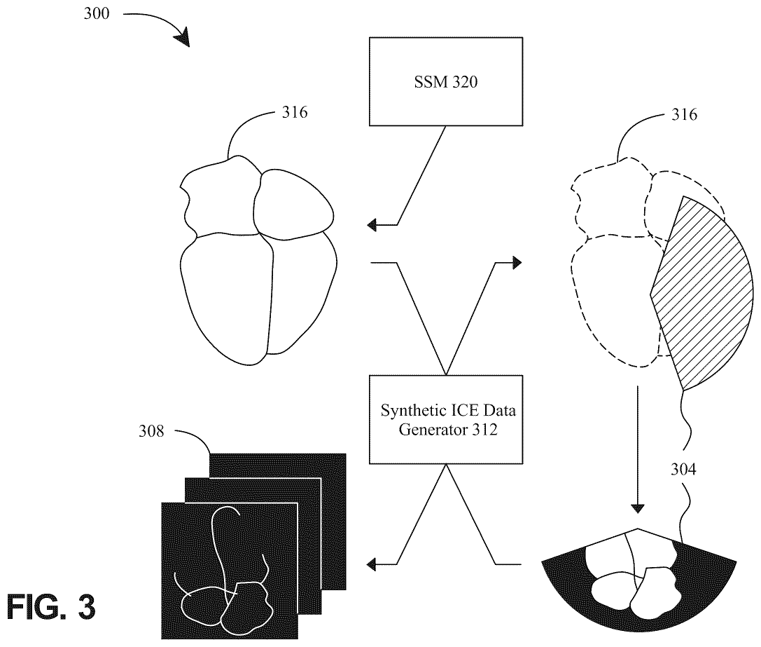

Now referring to FIG. 3, a flow diagram of an exemplary embodiment of an ICE example generation process 300. In an embodiment, cardiac anatomy training data may be generated, at least in part, via ICE example generation process 300. In some cases, processor may be configured to receive a 3D model of the heart, such as, without limitation, template model, initial model, subsequent 3D model, and/or any 3D model of cardiac anatomy 316 and identify an ICE view 304 based on the received 3D model. As used herein, “cardiac anatomy,” refers to the structural composition of the heart and its associated blood vessels. In some cases, 3D model received by processor may be derived from CT scans as described above with reference to FIG. 1. In other cases, processor may receive CT scans directly instead of 3D models. A synthetic ICE frame 308 may then be generated, by processor, as a function of identified ICE view 304, wherein the synthetic ICE frame may be used as one or the training examples in cardiac anatomy training data.

With continued reference to FIG. 3, in some cases, processor may interface with one or more 3D models (i.e., detailed representation of heart's anatomy in a 3D space, capturing intricate structures, chambers, vessels, valves, among others) as described above, or other imaging modalities and/or databases, and equipped with algorithms e.g., CNN, gradient boosting machines, SVM, PCA, and/or the like to analyze model's geometry and spatial relationships upon receiving the 3D models. In some cases, 3D models may be received from SSM 320 via a communicative connection between processor and SSM 320. As used in this disclosure, a “statistical shape model (SSM)” is a data structure representing, including, and/or utilizing a mathematical model that captures principal modes of variation in shape across a population of cardiac anatomies. In some cases, SSM 320 may be constructed by analyzing one or more datasets of shapes and identifying, for example, mean shapes and main modes of variation within the one or more datasets. In a non-limiting example, SSM 320 may start with calculation of at least one mean shape, which represents an average geometry of all the heart shapes in a given dataset, wherein the at least one mean shape may be served as a central reference point for processor to understand different variations. In some cases, dataset may include, without limitation, cardiac anatomy training data, cardiac geometry training data, and/or any datasets within ICE image databases. SSM 320 may also identify one or more principal modes of variation within given datasets described herein, wherein the “principal modes of variations,” for the purpose of this disclosure, refer to main patterns or directions along which data points vary within dataset. In a non-limiting example, identifying principal modes of variations may include applying principal component analysis (PCA) on given dataset. Additionally, or alternatively, shapes may be described directly using plurality of shape parameter sets (in cardiac geometry training data). In some cases, shape parameter sets may correspond to a plurality of modes of variations. Further, one or more statistical constraints (e.g., mean, variance, correlation, boundary, proportion constraint and/or the like) may be introduced into SSM 320 based on the distribution of shape parameters within plurality of shape parameter sets.

In a non-limiting example, processor may be configured to determine an optimal viewpoints or angles from which ICE view 304 would provide a desired diagnostic value or procedural guidance.

Still referring to FIG. 3, in some cases, identification and selection of ICE view 304 may be automatically identified, using one or more machine learning models as described herein. In a non-limiting example, processor may utilize one or more machine learning models trained on cardiac anatomy 316 viewpoints identification training data, wherein the cardiac anatomy 316 viewpoints identification training data may include a plurality of cardiac anatomies as input correlated to a plurality of ICE images as output and identify at least one ICE view 304 (most informative) for a given cardiac anatomy 316 using the trained machine learning models.

Still referring to FIG. 3, in other cases, ICE view 304 may be defined by a user such as a medical professional. In a non-limiting example user interface of display device may allow a user (e.g., a clinician) to manually rotate, pan, and zoom displayed 3D model and/or corresponding CT scans. As user do so, processor may dynamically calculate and displays potential ICE views 304 based on user's chosen perspective. Additionally, or alternatively, depending on cardiac procedure being planned or executed, processor may prioritize certain ICE views 304. For instance, and without limitation, ICE view 304 may be pre-defined. For atrial fibrillation ablation, ICE view 304 may showcase the pulmonary veins' entrances into the LA may be emphasized. In other cases, ICE view 304 may be automatically identified, by processor, using one or more machine learning models as described herein, such as, without limitation, synthetic ICE data generator as described in detail below.

With continued reference to FIG. 3, as used in this disclosure, a “synthetic ICE frame” refers to a digitally generated or simulated image that emulates a visual representation obtained from ICE view 304. In some cases, synthetic ICE frames 308 may be produced using computational methods and/or models such as, without limitation, a synthetic ICE data generator 312 based on pre-existing data, models, or simulations e.g., identified ICE views 304. In a non-limiting example, synthetic ICE frames 308 may include a simplified version e.g., an image illustrating heart anatomy via a plurality of lines indicating contours of heart's structure as shown in FIG. 3. One or more image processing techniques and/or computer vision algorithms such as, without limitation, histogram equalization, adaptive filtering, edge detection (e.g., Canny or Sobel operators), contour extraction, and/or the like may be applied, by processor, on a segmented CT scan and/or 3D models based on identified ICE view 304. Synthetic ICE frame 308 may be rendered on a blank canvas or background that mimics the echogenicity of an ICE image according to extracted contours, wherein the extracted contours may be represented as a bold lines and enhanced with shading to give depth. In some cases, synthetic ICE frame 308 may be validated and verified by overlaying synthetic ICE frame 308 onto original ICE view 304, ensuring accuracy and resemblance.

Still referring to FIG. 3, in some cases, generating synthetic ICE frames 308 may include implementations of one or more aspects of “generative artificial intelligence (AI),” a type of A l that uses machine learning algorithms to create, establish, or otherwise generate data such as, without limitation, ICE images, ICE videos, and/or the like that is similar to one or more provided training examples. In an embodiment, machine learning module described herein may generate one or more generative machine learning models that are trained on one or more set of CT scans and/or 3D models in ICE image view 304 as described above. Synthetic ICE data generator 312 may include one or more generative machine learning models may be configured to generate new examples that are similar to the training data of the one or more generative machine learning models but are not exact replicas; for instance, and without limitation, data quality or attributes of the generated examples may bear a resemblance to the training data provided to one or more generative machine learning models, wherein the resemblance may pertain to underlying patterns, features, or structures found within the provided training data.

Still referring to FIG. 3, in some cases, generative machine learning models within synthetic ICE data generator may include one or more generative models. As described herein, “generative models” refers to statistical models of the joint probability distribution P(X,Y) on a given observable variable x, representing features or data that can be directly measured or observed (e.g. CT scans and/or 3D models derived from CT scans) and target variable y, representing the outcomes or labels that one or more generative models aims to predict or generate (e.g., synthetic ICE frames 308). In some cases, generative models may rely on Bayes theorem to find joint probability; for instance, and without limitation, Naïve Bayes classifiers may be employed by computing device to categorize input data such as, without limitation, CT scans and/or 3D models derived from CT scans into different views.

In a non-limiting example, and still referring to FIG. 3, one or more generative machine learning models may include one or more Naïve Bayes classifiers generated, by processor, using a Naïve bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor may then calculate a likelihood table by calculating probabilities of different data entries and classification labels. Processor may utilize a naïve Bayes equation to calculate a posterior probability for each class. A class containing the highest posterior probability is the outcome of prediction.

Still referring to FIG. 3, although Naïve Bayes classifier may be primarily known as a probabilistic classification algorithm; however, it may also be considered a generative model described herein due to its capability of modeling the joint probability distribution P(X, Y) over observable variables X and target variable Y. In an embodiment, Naïve Bayes classifier may be configured to make an assumption that the features X are conditionally independent given class label Y, allowing generative model to estimate the joint distribution as P(X, Y)=P(Y)ΠiP(Xi|Y), wherein P(Y) may be the prior probability of the class, and P (Xᵢ|Y) is the conditional probability of each feature given the class. One or more generative machine learning models containing Naïve Bayes classifiers may select a class label y according to prior distribution P(Y), and for each feature Xᵢ, sample at least a value according to conditional distribution P(Xᵢ|y). Sampled feature values may then be combined to form one or more new data instance with selected class label y. In a non-limiting example, one or more generative machine learning models may include one or more Naïve Bayes classifiers to generate new examples of ICE images based on CT scans and/or 3D models derived from CT scans (e.g., identified ICE views 304), wherein the models may be trained using training data containing a plurality of features of input data as described herein and/or the like correlated to a plurality of ICE views.

Still referring to FIG. 3, in some cases, one or more generative machine learning models may include generative adversarial network (GAN). As used in this disclosure, a “generative adversarial network” is a type of artificial neural network with at least two sub models (e.g., neural networks), a generator, and a discriminator, that compete against each other in a process that ultimately results in the generator learning to generate new data samples, wherein the “generator” is a component of the GAN that learns to create hypothetical data by incorporating feedbacks from the “discriminator” configured to distinguish real data from the hypothetical data. In some cases, generator may learn to make discriminator classify its output as real. In an embodiment, discriminator may include a supervised machine learning model while generator may include an unsupervised machine learning model.

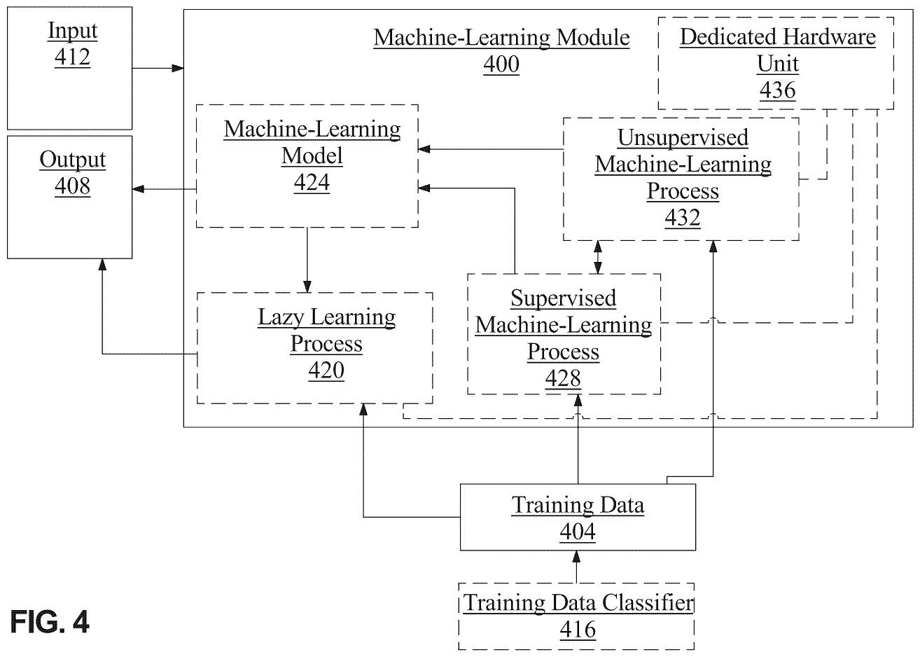

With continued reference to FIG. 3, in an embodiment, discriminator may include one or more discriminative models, i.e., models of conditional probability P(Y|X=x) of target variable Y, given observed variable X. In an embodiment, discriminative models may learn boundaries between classes or labels in given training data. In a non-limiting example, discriminator may include one or more classifiers as described in further detail below with reference to FIG. 4 to distinguish between different categories e.g., real vs. fake, or states e.g., TRUE vs. FALSE within the context of generated data such as, without limitations, synthetic ICE frames 308, and/or the like. In some cases, processor may implement one or more classification algorithms such as, without limitation, Support Vector Machines (SVM), Logistic Regression, Decision Trees, and/or the like to define decision boundaries.

In a non-limiting example, and still referring to FIG. 3, generator of GAN may be responsible for creating synthetic data that resembles real ICE images. In some cases, GAN may be configured to receive CT scans and/or 3D models derived from CT scans as input and generates corresponding examples of ICE images containing information describing heart anatomy in different ICE views. On the other hand, discriminator of GAN may evaluate the authenticity of the generated content by comparing it to true ICE images, for example, discriminator may distinguish between genuine and generated content and providing feedback to generator to improve the model performance. Additionally, or alternatively, GAN may include a conditional GAN as an extension of the basic GAN as described herein that allows for generation of ICE images using pre-existing CT scans and/or 3D models derived from CT scans based on certain conditions or labels. In standard GAN, generator may produce samples from random noise, while in a conditional GAN, generator may produce samples based on random noise and a given condition or label.

With continued reference to FIG. 3, in other embodiments, one or more generative models may also include a variational autoencoder (VA E). As used in this disclosure, a “variational autoencoder” is an autoencoder (i.e., an artificial neural network architecture) whose encoding distribution is regularized during the model training process in order to ensure that its latent space includes desired properties allowing new data sample generation. In an embodiment, VAE may include a prior and noise distribution respectively, trained using expectation-maximization meta-algorithms such as, without limitation, probabilistic PCA, sparse coding, among others. In a non-limiting example, VEA may use a neural network as an amortized approach to jointly optimize across input data and output a plurality of parameters for corresponding variational distribution as it maps from a known input space to a low-dimensional latent space. Additionally, or alternatively, VA E may include a second neural network, for example, and without limitation, a decoder, wherein the “decoder” is configured to map from the latent space to the input space.

In a non-limiting example, and still referring to FIG. 3, VAE may be used by processor to model complex relationships between CT scans and/or 3D models derived from CT scans. In some cases, VA E may encode input data into a latent space, capturing example ICE images. Such encoding process may include learning one or more probabilistic mappings from observed CT scans and/or 3D models derived from CT scans to a lower-dimensional latent representation. Latent representation may then be decoded back into the original data space, therefore reconstructing the 3D models representing example ICE images. In some cases, such decoding process may allow VA E to generate new examples or variations that are consistent with the learned distributions.

Additionally, or alternatively, and still referring to FIG. 3, processor may be configured to continuously monitor synthetic ICE data generator. In an embodiment, processor may configure discriminator to provide ongoing feedback and further corrections as needed to subsequent input data. An iterative feedback loop may be created as processor continuously receive real-time data, identify errors (e.g., distance between synthetic ICE frame 308 and real ICE images) as a function of real-time data, delivering corrections based on the identified errors, and monitoring subsequent model outputs and/or user feedbacks on the delivered corrections. In an embodiment, processor may be configured to retrain one or more generative machine learning models within synthetic ICE data generator based on user modified ICE frames or update training data of one or more generative machine learning models within synthetic ICE data generator by integrating validated synthetic ICE frames (i.e., subsequent model output) into the original training data. In such embodiment, iterative feedback loop may allow synthetic ICE data generator to adapt to the user's needs and performance requirements, enabling one or more generative machine learning models described herein to learn and update based on user responses and generated feedbacks.

With continued reference to FIG. 3, other exemplary embodiments of generative machine learning models may include, without limitation, long short-term memory networks (LSTMs), (generative pre-trained) transformer (GPT) models, mixture density networks (M DN), and/or the like. As an ordinary person skilled in the art, upon reviewing the entirety of this disclosure, will be aware of various generative machine learning models may be used generating synthetic ICE frames 308.

Still referring to FIG. 3, in a further non-limiting embodiment, synthetic ICE data generator 312 may be further configured to generate a multi-model neural network that combines various neural network architectures described herein. In a non-limiting example, multi-model neural network may combine LSTM for time-series analysis with GPT models for natural language processing. Such fusion may be applied by computing device to generate synthetic ICE frames 308. In some cases, multi-model neural network may also include a hierarchical multi-model neural network, wherein the hierarchical multi-model neural network may involve a plurality of layers of integration; for instance, and without limitation, different models may be combined at various stages of the network. Convolutional neural network (CNN) may be used for image feature extraction, followed by LSTM s for sequential pattern recognition, and a M DN at the end for probabilistic modeling. Other exemplary embodiments of multi-model neural network may include, without limitation, ensemble-based multi-model neural network, cross-modal fusion, adaptive multi-model network, among others. As an ordinary person skilled in the art, upon reviewing the entirety of this disclosure, will be aware of various generative machine learning models may be used to generating synthetic ICE frames 308 as described herein. As an ordinary person skilled in the art, upon reviewing the entirety of this disclosure, will be aware of various multi-model neural network and combination thereof that may be implemented by apparatus 100 in consistent with this disclosure.

Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm instantiated in hardware or software logic, data structures, and/or functions that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example inputs such as plurality of ultrasonic signals and at least a position signal and outputs such as movement vector.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data to historical computed tomography data corresponding to historical movement vectors.

Still referring to FIG. 4, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 4, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 4, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 4, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 4, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 4, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 4, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 4, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 4, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 4, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 4, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a medium value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{I Q R} .$$

Persons skilled in the art, upon revieing the entirety of this disclosure, will be aware of various alternative or additional approaches that may be used for feature scaling.

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 4, machine-learning processes as described in this disclosure may be used to generate machine-learning models 424. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include plurality of ultrasonic signals and at least a position signal as described above as inputs, movement vector as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

With further reference to FIG. 4, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 4, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 432 may not require a response variable; unsupervised processes 432 may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, A daBoost, gradient tree boosting, and/or voting classifier methods. Machine-learning algorithms may include neural net algorithms, including convolutional neural net processes.

Still referring to FIG. 4, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 4, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 4, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 4, one or more processes or algorithms described above may be performed by at least a dedicated hardware unit 436. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 436 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 436 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 436 to perform one or more operations described herein, such as evaluation of model and/or algorithm outputs, one-time or iterative updates to parameters, coefficients, weights, and/or biases, and/or any other operations such as vector and/or matrix operations as described in this disclosure.

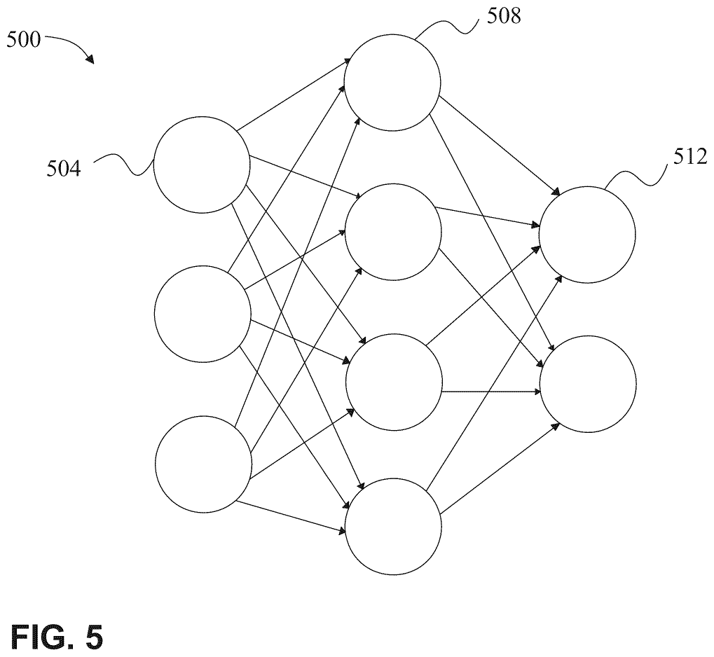

Referring now to FIG. 5, an exemplary embodiment of neural network 500 is illustrated. A neural network 500 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 504, one or more intermediate layers 508, and an output layer of nodes 512. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

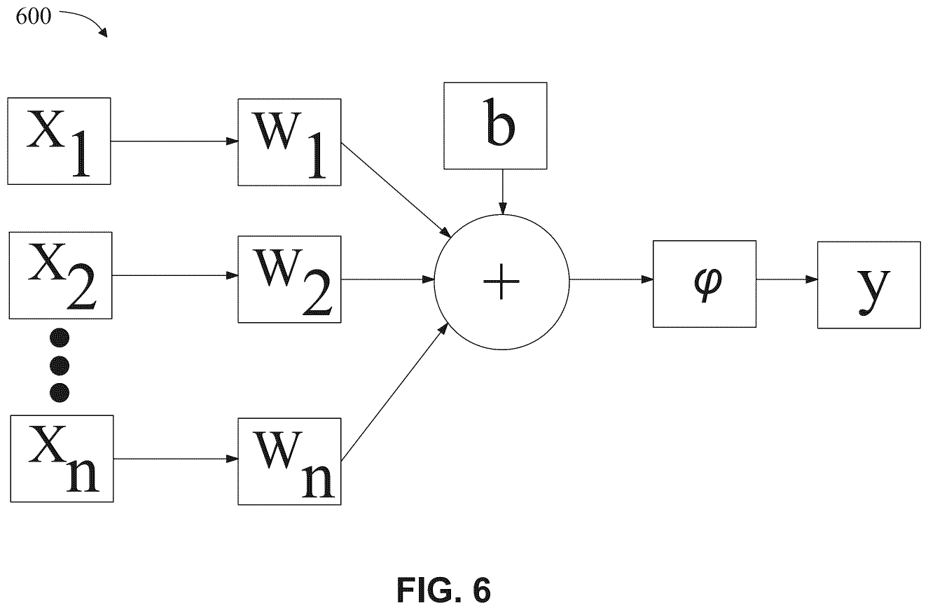

Referring now to FIG. 6, an exemplary embodiment of a node 600 of a neural network is illustrated. A node may include, without limitation, a plurality of inputs x; that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tan h (hyperbolic tangent) function, of the form

$$\frac{{e}^{x} - {e}^{- x}}{{e}^{x} + {e}^{- x}} ,$$

tan h derivative function such as ƒ(x)=tan h²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tan h(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input x; may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

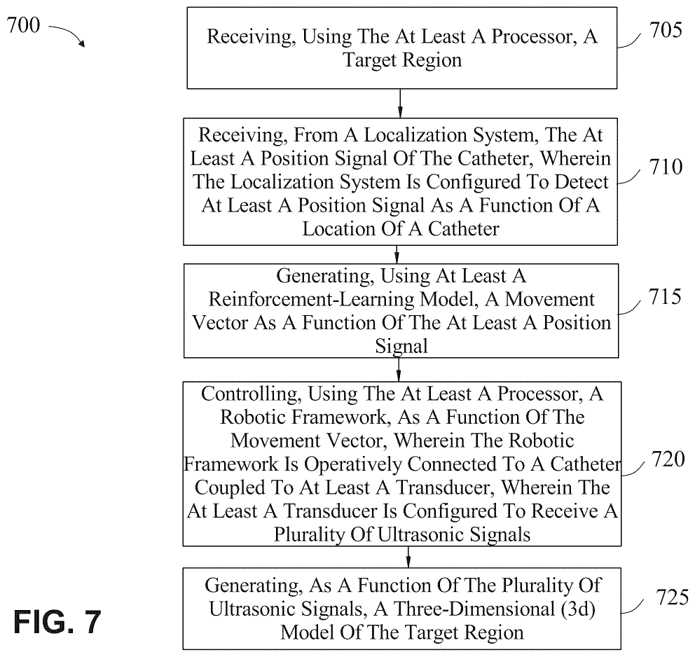

Referring now to FIG. 7, a flow diagram of an exemplary method 700 for controlling a robotic framework to perform imaging modalities is illustrated. At step 705, method 700 includes receiving, using the at least a processor, a target region. In an embodiment, the robotic framework further may include a robotic arm coupled to an end effector, wherein the robotic arm is configured to provide multi-axis movement relative to the target region. This may be implemented as described and with reference to FIGS. 1-6.

Still referring to FIG. 7, at step 710, method 700 includes receiving, from a localization system, the at least a position signal of the catheter, wherein the localization system is configured to detect at least a position signal as a function of a location of a catheter. This may be implemented as described and with reference to FIGS. 1-6.

Still referring to FIG. 7, at step 715, method 700 includes generating, using at least a reinforcement-learning model, a movement vector as a function of the at least a position signal. In an embodiment, the at least a reinforcement model may be trained on a computed tomography training dataset, wherein the computed tomography training dataset may include historical computed tomography data corresponding to historical movement vectors. In an embodiment, the movement vector may include one or more of a spatial variable and a temporal variable. In an embodiment, the at least a reinforcement-learning model may be trained as a function of iterative updates to parameters through interactions with an environment. In an embodiment, the at least a reinforcement-learning model may be trained using a supervised learning process, wherein the supervised learning process comprises training the at least a reinforcement-learning model on labeled position data representing historical catheter trajectory patterns. In an embodiment, the movement vector is dynamically adjusted in real-time based on feedback from the localization system and a comparison of the position signal to the target region. In an embodiment, the at least a reinforcement-learning model may be trained using a reinforcement learning process, wherein the reinforcement learning process may include optimizing a reward function based on a comparison of the 3D model to 3D medical imaging data. In an embodiment, the at least a reinforcement-learning model may be trained using a hybrid approach combining reinforcement learning and supervised learning, wherein the reinforcement learning optimizes the reward function, and the supervised learning refines an adherence of the reinforcement-learning model to known trajectory patterns. This may be implemented as described and with reference to FIGS. 1-6.

Still referring to FIG. 7, at step 720, method 700 includes controlling, using the at least a processor, a robotic framework, as a function of the movement vector, wherein the robotic framework is operatively connected to a catheter coupled to at least a transducer, wherein the at least a transducer is configured to receive a plurality of ultrasonic signals. This may be implemented as described and with reference to FIGS. 1-6.

Still referring to FIG. 7, at step 725, method 700 includes generating, as a function of the plurality of ultrasonic signals, a three-dimensional (3D) model of the target region. In an embodiment, the at least a processor may generate a visualization based on the 3D model of the target region and display the visualization using a display device. This may be implemented as described and with reference to FIGS. 1-6.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (eg., a computing device) and that causes the machine to perform any one of the methodologies and/or embodiments described herein. Examples of a machine-readable storage medium include, but are not limited to, a magnetic disk, an optical disc (eg., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (eg., data structures and data) that causes the machine to perform any one of the methodologies and/or embodiments described herein.

Examples of computing device include, but are not limited to, an electronic book reading device, a computer workstation, a terminal computer, a server computer, a handheld device (eg., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

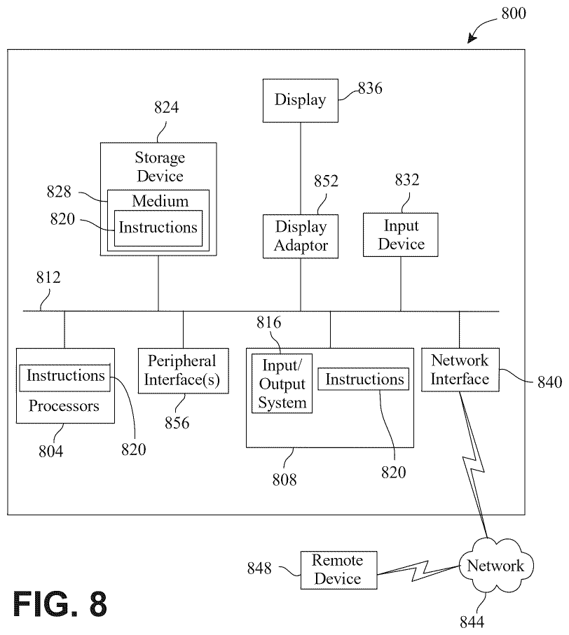

FIG. 8 shows a diagrammatic representation of one embodiment of computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (eg., stored on one or more machine-readable media) instructions (eg., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (eg., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (eg., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (eg., a keyboard), a pointing device, a joystick, a gamepad, an audio input device (eg., a microphone, a voice response system, etc.), a cursor control device (e.g., a mouse), a touchpad, an optical scanner, a video capture device (eg., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display device 836, discussed further below. Input device 832 may be utilized as a user selection device for selecting one or more graphical representations in a graphical interface as described above.

A user may also input commands and/or other information to computer system 800 via storage device 824 (eg., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (eg., the Internet, an enterprise network), a local area network (e.g., a network associated with an office, a building, a campus or other relatively small geographic space), a telephone network, a data network associated with a telephone/voice provider (eg., a mobile communications provider data and/or voice network), a direct connection between two computing devices, and any combinations thereof. A network, such as network 844, may employ a wired and/or a wireless mode of communication. In general, any network topology may be used. Information (eg., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for controlling a robotic framework to perform imaging modalities, wherein the apparatus comprises:
a robotic framework, wherein the robotic framework is operatively connected to a catheter coupled to at least a transducer, wherein the at least a transducer is configured to receive a plurality of ultrasonic signals;
at least a localization system configured to detect at least a position signal as a function of a location of the catheter, wherein the localization system comprises an ultrasound-based localization system, wherein the ultrasound-based localization system is configured to use the at least a transducer to determine distance in real time as a function of an amount of time for at least an ultrasound wave to reach at least a receiver; and
at least a computing device communicatively connected to the at least a transducer, wherein the at least a computing device comprises:
a memory; and
at least a processor communicatively connected to the memory, wherein the memory contains instructions configuring the at least a processor to:
receive a target region;
receive, from the localization system, the at least a position signal of the catheter;
generate, using at least a reinforcement-learning model, a movement vector as a function of the at least a position signal, wherein the reinforcement-learning model has been trained in a simulated environment using simulated ultrasonic sweeps derived from computed tomography (CT) data;
control the robotic framework, as a function of the movement vector; and
generate, as a function of the plurality of ultrasonic signals, a three-dimensional (3D) model of the target region.

2. (canceled)

3. The apparatus of claim 1, wherein the at least a processor is further configured to:
generate a visualization based on the 3D model of the target region; and
display the visualization using a display device, wherein the display device is configured to allow a user to manually rotate the 3D model.

4. The apparatus of claim 1, wherein the movement vector comprises one or more of a spatial variable and a temporal variable.

5. The apparatus of claim 1, wherein the robotic framework further comprises a robotic arm coupled to an end effector, wherein the robotic arm is configured to provide multi-axis movement relative to the target region.

6. The apparatus of claim 1, wherein the at least a reinforcement-learning model is further trained using a reward function configured to provide feedback in a form of rewards or penalties.

7. The apparatus of claim 6, wherein the reward function assigns a positive reward to the at least a reinforcement-learning model based on the location of the catheter and the target region.

8. The apparatus of claim 1, wherein the movement vector is dynamically adjusted in real-time based on feedback from the localization system and a comparison of the at least a position signal to the target region.

9. The apparatus of claim 1, wherein the at least a reinforcement-learning model has been trained using a reinforcement learning process, wherein the reinforcement learning process comprises optimizing a reward function based on a comparison of the 3D model to 3D medical imaging data.

10. The apparatus of claim 9, wherein the at least a reinforcement-learning model is trained using a hybrid approach combining reinforcement learning and supervised learning, wherein the reinforcement learning optimizes the reward function, and the supervised learning refines an adherence of the reinforcement-learning model to known trajectory patterns.

11. A method for controlling a robotic framework to perform imaging modalities, wherein the method comprises:
receiving, using at least a processor, a target region;
receiving, from a localization system, the at least a position signal of a catheter, wherein the localization system is configured to detect at least a position signal as a function of a location of the catheter, wherein the localization system comprises an ultrasound-based localization system, wherein the ultrasound-based localization system is configured to use at least a transducer determine distance in real time as a function of an amount of time for at least an ultrasound wave to reach at least a receiver;
generating, using at least a reinforcement-learning model, a movement vector as a function of the at least a position signal, wherein the reinforcement-learning model has been trained in a simulated environment using simulated ultrasonic sweeps derived from computed tomography (CT) data;
controlling, using the at least a processor, a robotic framework, as a function of the movement vector, wherein the robotic framework is operatively connected to the catheter coupled to the at least a transducer, wherein the at least a transducer is configured to receive a plurality of ultrasonic signals; and
generating, as a function of the plurality of ultrasonic signals, a three-dimensional (3D) model of the target region.

12. (canceled)

13. The method of claim 11, further comprising:
generating, using the at least a processor, a visualization based on the 3D model of the target region; and
displaying the visualization using a display device, wherein the display device is configured to allow a user to manually rotate the 3D model.

14. The method of claim 11, wherein the movement vector comprises one or more of a spatial variable and a temporal variable.

15. The method of claim 11, wherein the robotic framework further comprises a robotic arm coupled to an end effector, wherein the robotic arm is configured to provide multi-axis movement relative to the target region.

16. The method of claim 11, wherein the at least a reinforcement-learning model is further trained using a reward function configured to provide feedback in a form of rewards or penalties.

17. The method of claim 16, wherein the reward function assigns a positive reward to the reinforcement-learning model based on the location of the catheter and the target region.

18. The method of claim 11, wherein the movement vector is dynamically adjusted in real-time based on feedback from the localization system and a comparison of the at least a position signal to the target region.

19. The method of claim 11, wherein the at least a reinforcement-learning model has been trained using a reinforcement learning process, wherein the reinforcement learning process comprises optimizing a reward function based on a comparison of the 3D model to 3D medical imaging data.

20. The method of claim 19, wherein the at least a reinforcement-learning model is trained using a hybrid approach combining reinforcement learning and supervised learning, wherein the reinforcement learning optimizes the reward function, and the supervised learning refines an adherence of the reinforcement-learning model to known trajectory patterns.
